(19) United States
(12) Patent Application Publication
(10) Pub. No.: US 2002/0103344 A1
(43) Pub. Date: Aug. 1, 2002

(54) Glycoprotein mutants of retrovirus envelopes and their biological applications

(76) Inventors: Francisco Veas, Mauguio (FR); Franz Jansen, Assas (FR); Dorothee Misse, Montpellier (FR)

Correspondence Address: NIXON & VANDERHYE P.C., 8th Floor, 1100 North Glebe Road, Arlington, VA, 22201-4714, US

(21) Appl. No.: 09/960,653

(22) Filed: Sep. 24, 2001

Related U.S. Application Data
(63) Continuation of application No. 09/647,395, filed on Dec. 4, 2000, now abandoned.
Application No. 09/647,395 is a 371 of international application No. PCT/FR99/00767, filed on Apr. 1, 2001.

(30) Foreign Application Priority Data
Apr. 1, 1998 (FR) 98/04056

(51) Int. Cl. A61K 39/12; A61K 39/21; C07K 2/00; C07K 4/00; C07K 5/00; C07K 7/00; C07K 14/00; C07K 16/00; C07K 17/00; A61K 38/00; C12P 21/08

(52) U.S. Cl. 530/388.7; 530/388.3; 530/324; 530/300; 530/388.75; 530/389.1; 424/208.1; 424/204.1

(57) ABSTRACT

The invention concerns retrovirus envelope glycoprotein mutants characterized in that they are glycoproteins capable of specifically binding with chemokine receptors and having an inhibiting activity with respect to a retroviral infection.

The subject of the invention is glycoprotein mutants of retrovirus envelopes and their biological applications.

It is known that, with HIV-1, the infection of target cells (monocytes or lymphocytes) is mediated by the envelope glycoproteins, gp120 and gp4l, with a very strong affinity bonding of gp120 to the CD4 receptor.

Gp120 has been subjected to various deletions and point mutations so as to identify the sites that participate in the association with the CD4.

It is apparent, on the basis of various work, that the trytophan in position 432 (according to the numbering system of Cordonnier et al., 1989, Nature 340:571-574) or in position 427, according to Kwong et al., 1998, Nature, 393:648), situated in the 4^{th} constant region C4, is critical for the bonding to CD4.

The bonding of gp120 to CD4 induces conformational changes in the envelope glycoproteins of HIV-1 which must encourage subsequent steps for the entry of the virus.

Desjardins et al, in J. of Cellular Biochemistry, n° suppl. 21b, p.229, 1995, disclose immunologic analysis works on gp120 deleted in their conserved and target regions in order to better expose the gp120 region involved in the CD4 binding.

Thali et al, in J. of Virology, July 1993, vol. 67, n °7, p.3978-3988, disclose the characterization of the recognition of regions involved in the interactions with CD4 by monoclonal antibodies and note that mutations in gp120 Cl regions have no effet on the recognition by said antibodies.

Recently, fusogenic co-receptors have been identified. Hence, the important role played by receptors belonging to the family of chemokines with several transmembrane passages with respect to HIV-1, HIV-2 and SIV have been reported.

It is apparent that distinct tropisms of various HIV colonies result from the targeting of various chemokine receptors and research carried out with recombinant glycoproteins of HIV-1 have shown the central role played by the V3 loop of the gp12O of HIV- 1 with regard to the tropism towards macrophages and the formation of syncytium or the fusion of CD4- positive lymphocytes in cultures.

The receptor CXCR4, namely fusine, operates as a co-receptor of HIV colonies having tropism with respect to T cells or T-cell line adapted (TCLA) cells.

A conformation change of the gp12O would be necessary to obtain correct bonding of HIV to the chemokines and would lead to the formation of an association of 3 molecules, with CXCR4 and the gp12O/CD4 complex.

In their work on the interactions between HIV-1 and the target cells, the attention of inventors continues to be drawn to the strongly preserved regions among the gp120 of various colonies of HIV-1, and also to the family of retroviruses comprising, in addition to HIV-1 and HIV-2, SIV, HTLV-1, VISNA, EIAV, VLB and VSR. These preserved structures often show the structure of an a helix (abbreviated to HXα).

The low stringency targeting, with an antibody, of a bank of combinatory peptides has enabled one to confirm that the Cl domain of gp12O contains an α helix, designated HXα1. This α-l helix corresponds to the helix structure that is the most important, compared to other a helix structures in gp12O. It is located at the interface between adjacent molecules of gpl2O in an oligomeric complex.

Taking these different disclosures inzo account, the inventors have sought to study the effects which could result from the deletion of these helix structures and particularly HXα-1 and have produced corresponding deleted glycoproteins.

The work which has been carried out has shown that such deletions modify the properties of bonds to the receptors and co-receptors of the glycoproteins on the target cells, and confer on them properties which are of great interest in the fight against infection by a retrovirus.

Advantageously, the results obtained with the HIV envelope glycoproteins may be verified with other retroviral glycoproteins and therefore permit the development of tools of major importance to prevent and treat retroviral infections.

Hence the aim of the invention is to provide new glycoprotein mutants of retrovirus envelopes, capable particularly of interacting in a specific way with receptors of the chemokine type and of exerting an inhibitory effect against a viral infection.

It is also aimed at the biological applications of these mutants.

The mutants of the invention are characterized in that they are glycoproteins capable of interacting specifically with the chemokine receptors and having an inhibiting activity with regard -tom a retroviral infection.

These specific chemokine mutants are characterized in that their activities particularly their inhibiting activity are independent of the CD4.

Mutants of the invention are characterized in that they are glycoproteins of retrovirus envelopes lacking at least one structure in an a-helix, such as is present in the native glycoproteins of an envelope.

The invention targets particularly those mutants in which the glycoproteins are lacking the α-1 helix such- as that present in the Cl region of the gp120 of HIV.

The amino acid sequence of these mutants therefore corresponds, with the exception of the deleted helical structure, to that of a native glycoprotein of an envelope, either complete or partial, comprising, in the latter case, at least the regions involved in the recognition of chemokine co-receptors on the target cells.

As a variant, the amino acid sequence of the mutants corresponds, with the exception of the deleted helical structure, to a sequence that is homologous to such a complete or partial sequence of a native glycoproteln of an envelope. In the traditional way, the homology is defined as when there is about at least 70% similarity between sequences, the modifications, deletions or mutations not affecting the properties observed with the reference sequence.

The reference to the envelope glycoproteins of a retrovirus or to the native glycoproteins, in the description and the claims therefore include the homologous sequences. It also includes the recombinant glycoproteins as expressed by vectors or systems of expression such as a baculovirus, on the surface of transfected cells.

The invention specially targets mutants in which the glycoproteins are lacking the fragment corresponding to the sequence (E61 to S85, according to the numbering system of Cordonnier et al. described above) as present -n te Cl region of the HIV gpl2O.

In an advantageous way, the mutants of the At invention constitute prototype viral envelopes for the search for immunogenic power in mammals, in particular, in primates.

Hence, the invention also targets the antibodies formed against the mutants of the invention.

The mutants of the invention are additionally of interest as competitors of drugs that are able to use CXCR4 as a cell target, permitting the selection of antiviral drugs.

They are also useful in the search for antibodies which recognize the same cellular receptor, with the purpose of inhibiting a viral infection by competition.

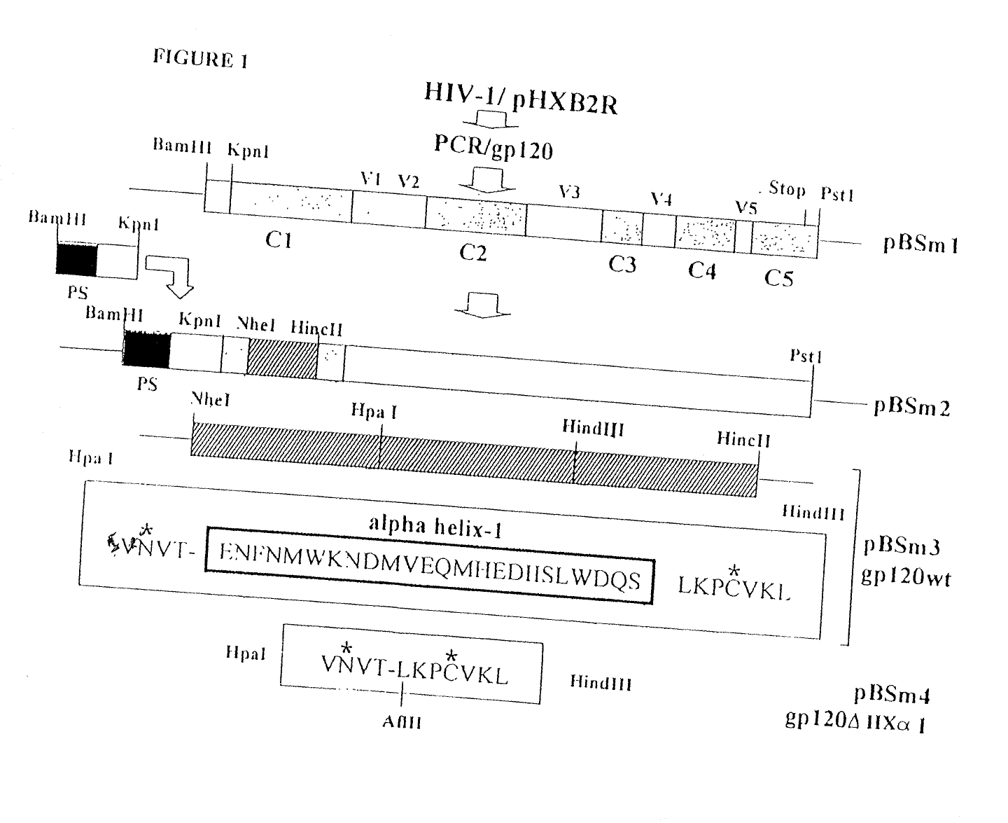

Other characteristics and advantages of the invention are given in the examples which follow, in which reference is made to FIGS. 1 to 8.

FIG. 1 giving a diagrammatic representation of a method of obtaining a mutant of gpl2O according to the invention,

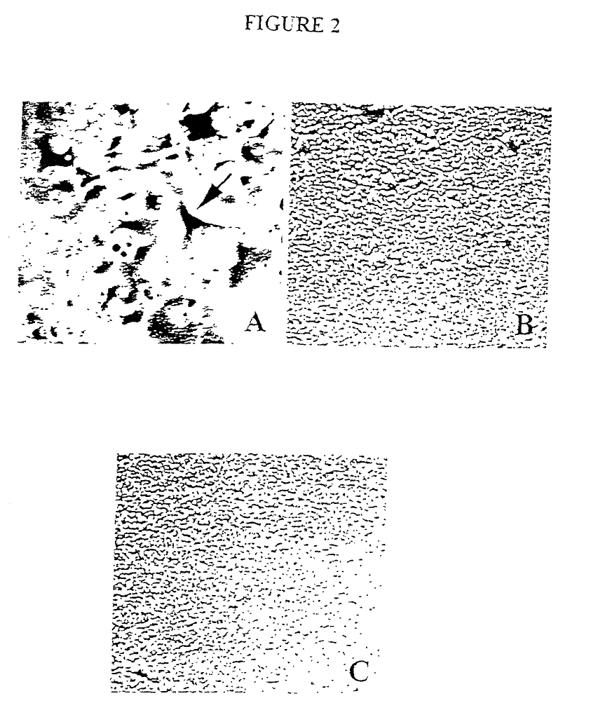

FIG. 2, phoros of co-cultures of HeLa-Tat and HeLa-P4 comprising genes CD4 and Lac-Z, the cultures of HeLa-Tat being transfected with a gpl2O of the wild type (gp120 wt), or a mutant according to the invention, non-transfected cell cultures being used as a control,

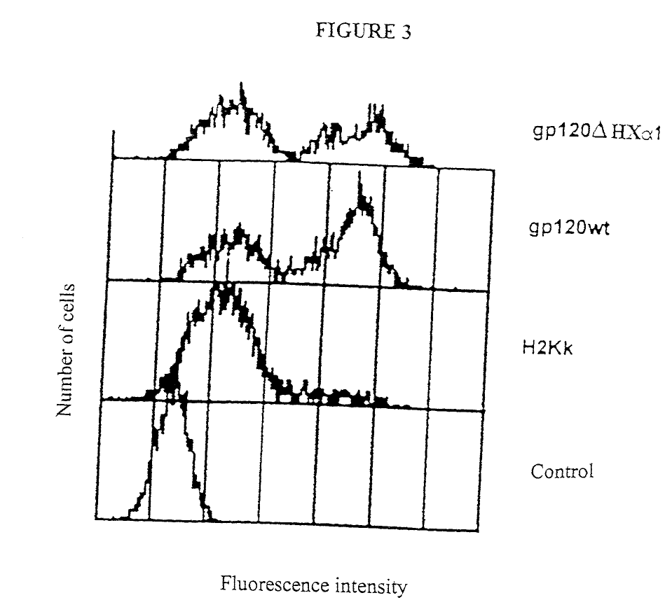

FIG. 3, the results of fluorescence measurements obtained with HeLa-Tat cells co-transfected by a plasmid expressing a membrane molecule and a plasmid expressing either gpl2O wt, or a mutant of gp120 according to the invention,

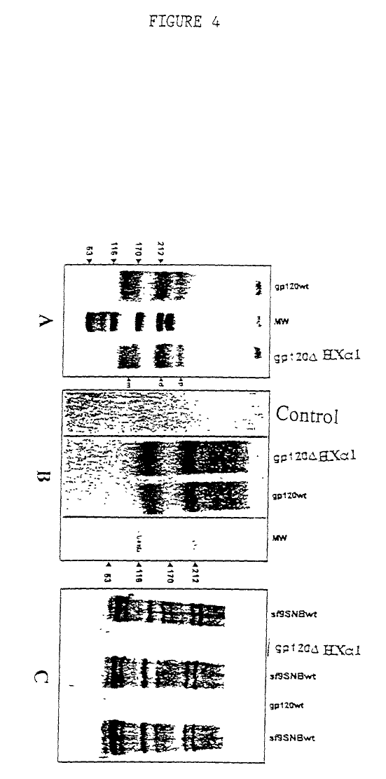

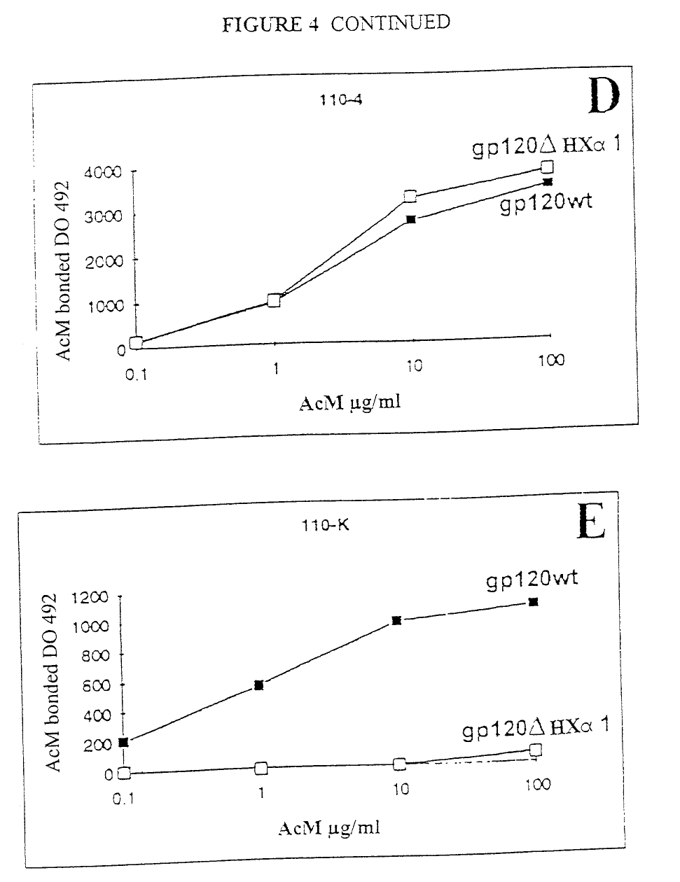

FIG. 4, the recombinant wt gp120 and mutant separated by SDS-PAGE,

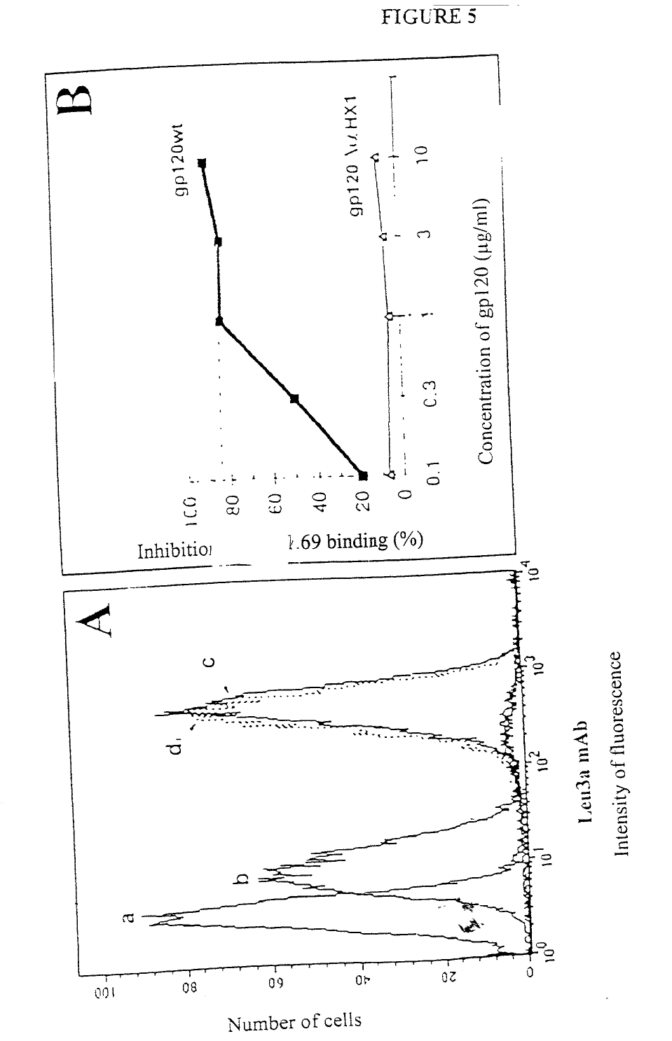

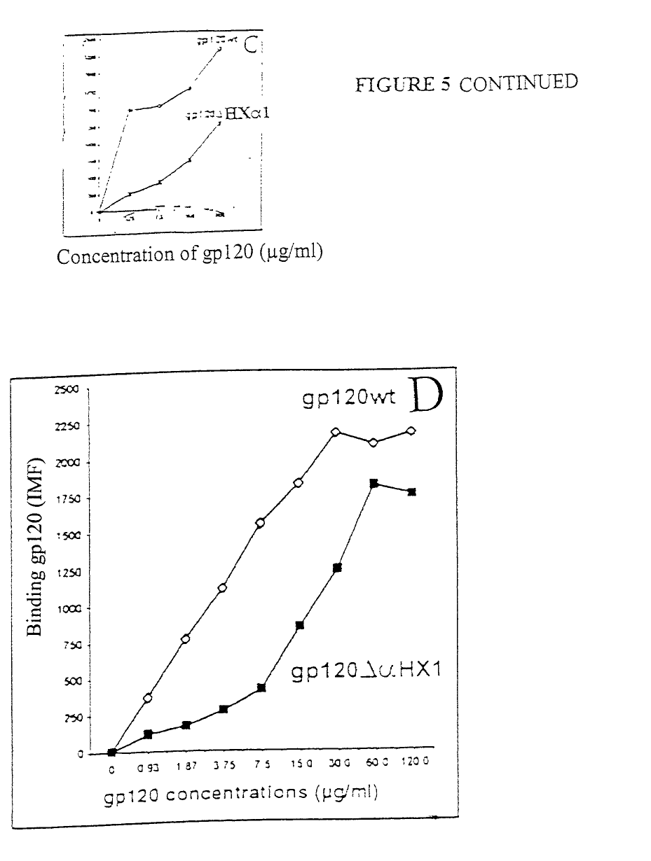

FIG. 5, the results of FACS analyses, that is to say with a cell separator by fluorescence and cellular ELISA relating to the effects of gp120 wt and a mutant according to the invention on the bonding to the surface of CEM cells of monoclonal antibody, anti-CD4,

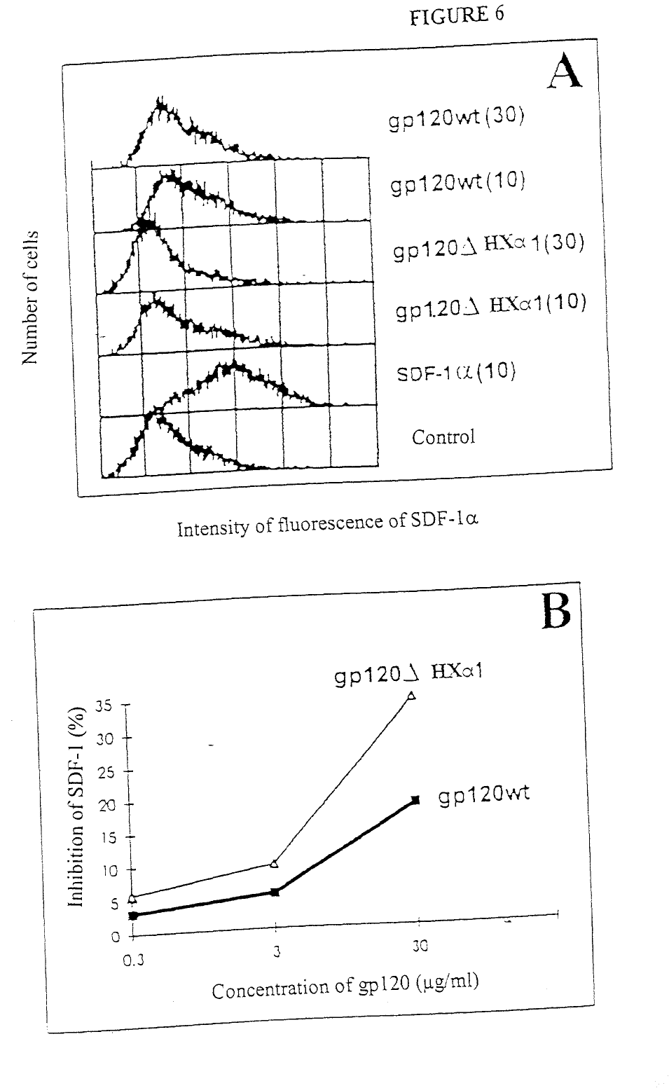

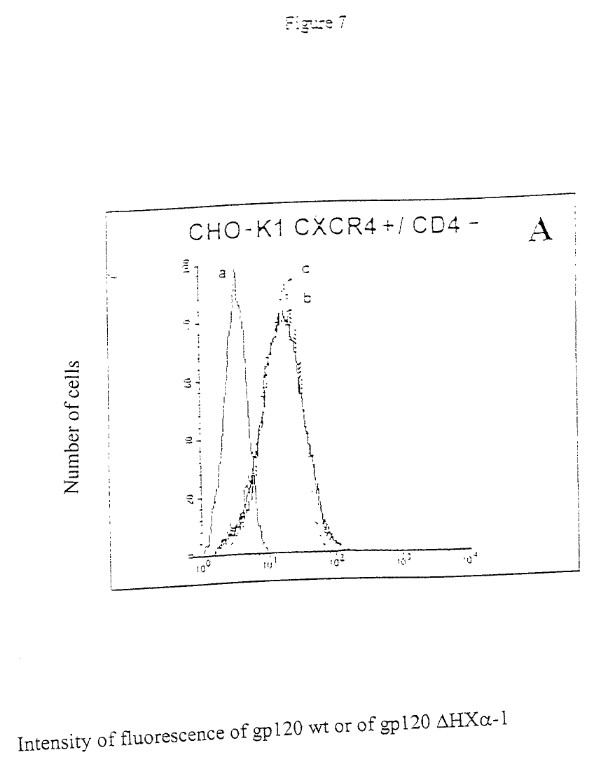

FIG. 6, the results of FACS analyses and cell-ELISA relating to the study of the bonding of SDF1-A to the CXCR4 in the presence of gp120 wt or a mutant of gp120 according to the invention,

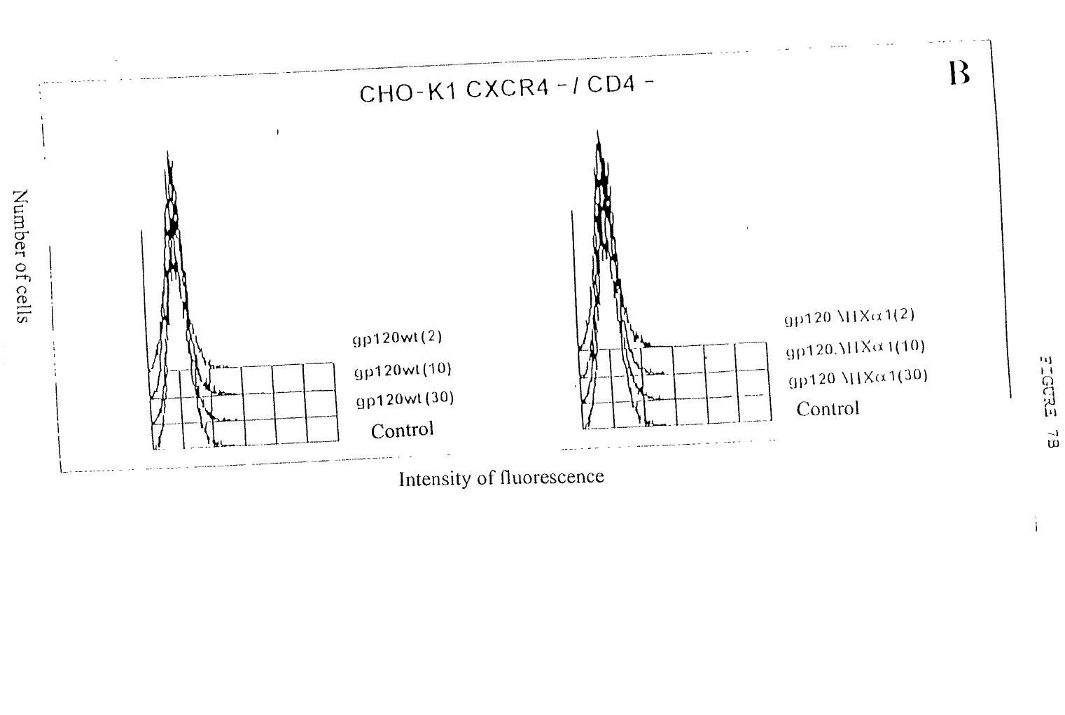

FIG. 7, the results of the FACS analysis relating to the bonding of the gpl2O wt and a mutant of gpl2O according to the invention to CHO—K1 cells CD4-negatives (CD4⁻), expressing a recombinant CXCR4 receptor (CXCR4⁻) , and CHO—K1 CD4⁻, CXCR4⁻cells, by way of a control, and

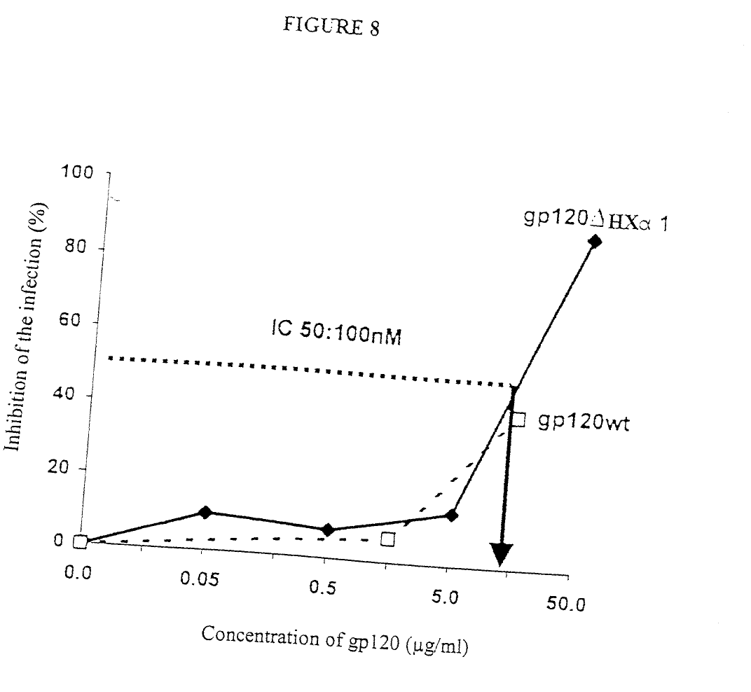

FIG. 8, the results of infection tests by HIV-1 of HeLa-P4 cells in the presence of gpl2O wt and gpl2O mutant according to the invention.

### EQUIPMENT USED IN THE EXPERIMENTS

Cell lines

The cell lines are held at 37° C. in a moist atmosphere including 5% of C0₂.

HeLa-P4 cells are used expressing the gene LacZ in a stable way, under the control of HIV-1 LTR (Cells HeLa CD4 LTR LacZ) (P. Charneau, Pasteur Institute). These cells are cultivated in a Dulbecco Modified Eagle Medium (DMEM) supplemented with 10% Fetal Calf Serum (FCS, Blomedla), 2 mM of L-glutam ne, penicillin, streptomycin, and 400 μg/ml of geneticin (G418).

The cell line HeLa-Tat (O. Schwartz, Pasteur Institute) is transfected in order to express the HIV-1 envelope and cultivated in a complete DMEM medium with 2 mM of methotrexate so as to select the positive clones Tat.

The cell line CEM CD4⁻, obtained from the ATCC is cultivated in a RPMI 1640 medium supplemented with 10% of FCS.

The recombinant cell line CHO—K1 (M. Parmentier, Euroscreen, Co, Brussels) is cultivated in a HamFl2 (Life Technologies), supplemented with 10% of FCS and 400 μg/ml of G418. The plasmid pHXB2R is a clone derived from HIV-1 IIIB (NIH, Bethesda, Aids Research and Reference Reagent Program, E.U.A.), and the colony HIV-1 LAI (H. Holmes, MRC, Aids Reagent Project, NIBSC, GB) is cultivated in the cell lines CEM and HeLa-CD4.

Antibodies

The monoclonal antibody 12G5 anti-CXCR4 (J. Hoxie, University of Pennsylvania, Philadelphia, U.S.A. or Pharmingen Dikinson) reacts specifically with the human CXCR4 protein and recognizes a conformational epitope, apparently located on the third extra-membrane loop of the molecule.

The HIV-immune immunoglobulin poor samples used, came from the N.I.H. (AIDS Reagent Repository) . They are mixtures of Ig from selected serums coming from seropositive persons.

The polyclonal sheep antibody D7324 is an anti-gp120 antibody developed against a peptide containing the amino acids from positions 497 to 511 of the gpl2O (Aalto BioReagents, Ireland).

The monoclonal antibody anti-gpl2O 110.4 is directed against the GPGR sequence of the V3 loop (Genetic Systems, U.S.A.) and the monoclonal antibody anti-gp120 110-K is directed against an epitope which is used to bond to the CD4 (Hydridolab, France).

The rabbit anti-serum anti-gpl2O was obtained by the inventors after immunization with a recombinant gpl2O from HIV-1 IIIB from Intracel Corp. (U.S.A.)

The monoclonal antibody Leu3a anti-CD4 is a product sold by Beckton and Dickinson (San Jose, Canada).

The monoclonal antibodies 13B8.2/1OKT4A and BL4/10KT4 anti-CD4 are products sold by Immunotech S.A. (Marseille, France).

The monoclonal antibodies ST4/F101.69 anti-CD4, ST40/F142.63 anti-CD4, BF5 anti-CD4, F93, 7G2 anti-CD100 and F145.GF3 anti-CD5 came from Sanofi (Montpellier, France). The monoclonal antibody OKT4 anti-CD4 is a product sold by Ortho Diagnostic Systems, Inc. (U.S.A.).

The anti-SDF1-α antibodies are products sold by R & D Systems (Great Britain).

Example 1: Method of obtaining a gpl2O mutant; cloning and expression of wild type gp120 .a; of the gp120 mutant ΔHXα-1

A diagram is shown in FIG. 1 illustrating the steps of the method used to remove the α-l helix structure from the Cl region of the gpl2O. In this diagram, the peptide signal (ps), the preserved regions (C1 to C5) and the variable regions (V1 to V5) are indicated.

The asterisks designate the sites of N-glycosylaticn and cystein preserved in the α-1 helix.

obtaining pBSm1

By PCR, a fragment of 1414 pb is amplified that codes for the gpl2O (amino acid V12 to R481), using the plasmid pHXB2R as a matrix and the following 2 starters

| | |
|---|---|
| direction starter: | 5′ GCAGGATCCGGTACCTGTGTGGAAGGAAGC 3′ |
| anti-direction starter: | 5′ GCACTGCAGTTAGCGTTTCTCTCTCTGCACCACTC 3′ |

The fragment generated contains a BamHI site upstream of the KpnI site of the gpl2O (V12) and a stop codon at the end of the sequence of the gpl2O followed by a Pstl site.

The BamHI-Pstl fragment is then cloned in a Bluescript® vector (pBS) (Stratagene) in which the KpnI site is removed by digestion with Kpnl, followed by a repair with the T4- DNA polymerase and by a religation, which leads to a sub-clone of gpl2O pBSm1.

Production of pBSm2

Next, the coding sequence for the N-terminal part of the gp12C (T1 to G11) is added and a new sequence of signal peptide, isolated from the gene ecdysteroid glycosyl transferase of the baculovirus of Autographa californica. To do this, overlapping oligonucleotides are used and are inserted into the BamHI-KpnI sites of oBSm1. The resulting plasmid is called pBSm2.

Production of pBSm3

2 unique restriction sites are introduced adjacent to the HXα-1 sequence using oligonucleotides which modify the codons V57-N58 and K91-L92, without modifying their ability to be coded.

According to the first modification, the codon GTA of V57 is replaced by GTT and the codon AAT of N58 by AAC. Regarding the second modification, the codon AA of K91 is replaced by AAG and the codon TTA of L92 by CTT, which creates respectively sites HpaI and HindIII.

Next the sequence is inserted into pBSm2 which leads to the plasmid pBSm3 being obtained.

Production of pBSm4

To remove the HXα-1 sequence, the wild type fragment HpaI-HindIII is excised from the plasmid pBSm3 and replaced by a transferred HpaI-HindIII fragment.

This fragment is reconstituted using 2 overlapping oligonucleotides

5′ AACGTGACACTTAAGCCATGTGTAA 3′, and
5′ AGCTTTACACATGGCTTAAGTGTCACGTT 3′

and a A-IIII site is also inserted into the L86 codon by changing CTA into CTT so as to identify the transferred fragment, which leads to the plasmid pBSm4.

Cloning and expression of wild type gpl2O and ΔHXα-1 mutant

Next one proceeds to the excision of the fragment BamHI-Pstl, comprising the complete coding sequence for gp12C from pBSm3 or pBSm3, pBSm4. This fragment is cloned in the BgLII-Pstl sites of the transfer vector p119P of the baculovirus 210 (M.H. Ogliastro).

SF9 cells are co-transfected with the purified viral DNA coming from the modified baculovirus AcSLP1O and the DNA coming from the recombinant vector of gpl2O p119P.

The recombinant baculoviruses are purified on plates, using the standard methods.

The SF9 cells are infected at a density of 5=10⁵ cells/ml and at an infection multiplicity of 5 UFP/cell. The supernatant is recovered 6 days after the infection. The wild type gp12O or gp12O wt, and the deleted gp12O, or gp12O ΔHXα-1, are concentrated and immuno-purified with the antibody anti-gp12O D7324, previously made insoluble, on bromacetyl-Sepharose®.

So as to verify the purity of the products, the proteins are separated by applying a gradient of from 4 to 15% of Phastgel® sodium dodecyl sulfate, in a discontinuous buffer system (PhastSystem, Pharmacia).

The resolved protein bands are transferred electrophoretically onto nitrocellulose. After saturation, the prints are incubated with the suitably marked antibodies.

The complete sequences of gp12O wt or gp12O ΔHXα-1 are cloned in an envelope expression vector of HIV-1 pCEL/E160 under the control of the CMV promoter.

pCEL/El6O (Y. Boublik and M. Sitbon IGM) derives from an retroviral envelope expression vector by insertion of the envelope of HIV-1 LAI.

In order to obtain the expression of the envelope with the desired gp12O, the fragment KpnI—NheI is substituted in the original corresponding sequence of pCEL/E160, using a second restriction site NheI localized upstream of the stop codon derived either from pBSm3, or from pBSm4.

Example 2: Study of the cellular fusion between the envelope proteins of HIV-1 and target cells

Use of the expression of the gene LacZ to provide evidence of a fusion

HeLa-Tat cells are deposited on plates with 6 wells, with a flat bottom, at a concentration of 8×10⁴ cells per well.

After 24 hours, the cells are transfected with 1 μg of the expression vector pCEL/E160 using the reactant Lipofectamine (Gibco Life Sciences, U.S.A.) following the recommendations of the manufacturer.

The ability of different envelope glycoproteins to induce the fusion and the formation of syncytia is evaluated. In order to do this, a co-culture with HeLa-P4 cells (expressing the receptor CXCR4) is carried out the comprising, in a constitutive manner, the CD4 receptor and transformed with a LTR HIV-1 LacZ construction.

After 24h, when the co-cultures are confluent, they are washed with a phosphate buffer saline (PBS) fixed with glutaraldehyde at 0.5% for 10 minutes at ambient temperature, and washed twice with PBS.

The cellular mono-layers are colored through incubation with a solution of 5-bromo-4-chloro-3- indolyl-β-D-galacropyranoside (X-gal), for 2 hours at 37° C., and washed twice with PBS.

Fusion events between the HeLa-P4 and the cells which are effective in expressing the HIV-1 envelope and the transactivator Tat lead to the induction of the in situ expression of the promoter of the reporter gene lacZ. For every envelope glycoprotein tested, counting is carried out of the total number of foci colored blue per well.

FIG. 2 reports the results obtained with A) HeLa-Tar cells expressing the wt envelope (containing gp12O wt), B) HeLa-Tat cells expressing the envelope with no α-1 helix (containing gp12O ΔHXα-1) and C) non-transfected HeLa-Tat cells. The presence of blue syncytium is indicated by an arrow.

The transfection with the wild type envelope leads to a large number of syncytia with expression of β-galactosidase (1000 to 2000 foci/well). In contrast, no foci are observed after infection with the envelope of the mutant ΔHXα-1 (FIG. 2) or with the envelope of the Friend ecotropic murine leukemia virus (F-MuLv) which is only fusogenic for mouse or rat cells.

Analysis by flow cytometry of the surface expression of the envelope cells

The absence of observed fusogenic properties with the gp12O ΔHXα-1 leads to verification of whether the expression of the envelope at the surface of the cells was modified.

Experiments were therefore carried out to control the percentage of gp12O at the surface of transfected HeLa-Tat cells, by flow cytometry, proceeding as follows :

The pCEL/E160 expression vector, bearing either the gp120 wz or SU (for:sub-surface unit) or the gp12O ΔHXα-1, is co-transfected in HeLa-Tat cells with the pMACS-Kk plasmid, which expresses a truncated murine membrane molecule H-2Kk (Miltenyl Biotech Inc.).

The transfection is carried out by particle bombardment using a biolistic apparatus PDS/1000/He (BioRad).

In summary, a coating is deposited on 3 mg of gold beads of 1.5 pm diameter, the coating having 2.5 μg of mixed DNA containing 0.5 μg of pMACSKk and 2 μg of pCEL/E160. After 24 hours culture bombardment, the transfected cells are detached, washed, incubated for 1 hour at 4° C. with 80 μl of magnetic micro-beads coated with the monoclonal antibody ant-H2Kk. A positive selection of the cells expressing the protein H2Kk is carried out using RS+ columns by separation on the magnetic Vario-MiACS system and following the recommendations of the manufacturer (Miltenyl Biotec Inc., Auburn, Canada). The selected cells are incubated for 1 hour at 4° C., with 100 μg/ml of anti-HIV human polyclonal IgG.

The cells are then washed, colored with anti-human goat IgG conjugated to phyco-erythrine (PE) (50 μl of a 1/50 dilution; Immunotech, Marseilles, France) , for 1 hour at 4° C.

The cells are washed 3 times in PBS/0.3% of BSA before progressing to the flow cytometry analysis using a FACSort apparatus (Beckton & Dickinson) to measure the percentages of gp120 at the surface of the cells.

As negative controls, cells transfected by the vector PMACS Kk (H2Kk) alone (coloration with anti-HIV IgG and conjugated anti-human IgG-PF4 and HeLa-Tat cells co-transfected with a wt envelope plasmid and the vector pMACS Kk, but only colored with the anti-human secondary antibody conjugated to PE.

The results obtained are reported in FIG. 3 which gives the number of cells as a function of the intensity of fluorescence for the controls and the transfected cells.

A unimodal population is observed with a low fluorescence intensity (mean fluorescence intensity=15.1) after the coloration of the HeLa-Tat control cells.

With the cells transfected by an envelope expression vector of the wild type or of the deleted type, similar distributions are noted for the negative and positive populations.

In both cases, the positive population is detected at a mean fluorescence intensity of about 170.

Example 3: Study of the bonding of the recombinant proteins gp12O wt and gp12O ΔHXα-1 to the CEM CD4⁺cells

General method

All of the bonding experiments are carried out with 2×10⁵ CEM cells suspended in 50 μl of PBS/3% BSA containing the desired monoclonal antibody at the appropriate concentration.

After 1 hour of incubation under agitation at 37° C., the cells are washed twice in PBS/3% BSA and then an anti-mouse antibody IgG-FITC (Sigma) or an anti-human IgG-PE conjugate (Immunotech) or an anti-human IgG-FITC conjugate (Immunotech) or a streptavidine-PE conjugate (Sigma) is added, at a dilution of 1/50.

After 1 additional hour of incubation, with agitation, at ambient temperature, the cells are washed three times, put back into suspension in PBS and analyzed by flow cytometry (one color) using a FACSort (Beckton & Dickinson) with Lysis II software. Each point represents the acquisition of 10,000 relevant events.

Purification of the recombinant gp120

The recombinant gp12O glycoproteins of baculovirus being used are concentrated and irrmuno-purified as described above. The proteins are resolved by SDS-PAGE (gradient from 4 to 15%).

FIG. 4 gives photographs of the gels with A) the proteins (4 μg) colored with silver nitrate or transferred onto a nitrocellulose membrane and the immunological print then obtained, B) with a polyclonal anti-gp12O rabbit anti-serum or a polyclonal rabbit anti-serum formed against a supernatant from SF9 cells infected by a wt baculovirus (SF9SNBwt).

On each plate, the tracks corresponding to the gp120 wt, gp12O ΔHXα-1 and the SF9SNBwt and the molecular weight markers (MW) have been indicated.

The positions of the monomers (m), the dimers (d) and the polymers (p) of the gp12O are marked by arrows.

The reactivities of the antibodies 110.4 (D) ct 110.K(E) anzi-gp12O with the native wt gp12O (black squares) and the gp12O ΔHXα-l (white squares) were determined. All the results have been corrected taking into account a basic antibody absorption in the absence of gp120 (usually D0₄₉₂ less than 0.10.)

Examination of FIG. 4A shows thy presence of 2 major bands corresponding to monomers and dimers of soluble gp120 wt and gp120 ΔHXα-1, with a degree of purity greater than 95%.

The analysis of the immunoblots of the proteins produced by the baculovirus, with a polyclonal anti-gp12O rabbit anti-serum, confirms the presence of monomer and oligomer forms of gp12O (FIG. 4B).

The purity of the gp120 proteins is demonstrated by the absence of reactivity with a rabbit antibody directed against the supernatant arising from SF9 cells infected with a wild type baculovirus (FIG. 4C).

Then a check was made of whether the gp120 ΔHXα-1 protein was recognized by the monoclonal antibodies 110.4 and 110.K, anti-gp12O, which react, respectively with the loop V3 and conformational structures involved in the recognition of the CD4.

The results obtained show that the V3 loop is recognized in an equivalent way in the gp12O wt and the gp120 ΔHXα-1 (FIG. 4D). On the other hand, the monoclonal antibody directed against the conformational site for recognition of the CD4 does not appear capable of reacting with the gp12O wt (FIG. 4E).

Effect of the gp12O on the bonding of monoclonal antibodies anti-CD4 to the cellular surface

FACS Analysis

The ability of the soluble gp12O ΔHXα-1 to associate with the CD4 was evaluated by determining the degree of bonding of CD4 specific monoclonal antibodies, Leu3a and F101.69-PE, which react with the CDR2 loop in the first domain D1 of 4, after pre-incubation of the CEM cells with the wild type gp12O or gp12O ΔHXα-1 (10 μg/ml), in the presence of 0.02% sodium azide.

The results of the FACS analysis are given in FIG. SA. In this FIG., a) to d) correspond respectively to the following conditions :

a) incubation of CEM cells with a goat -FITC anti-mouse IgG conjugate, as a negative control,

b) bonding of the Leu3a antibody, after incubation with gp120 wt,

c) bonding of the Leu3a antibody, after incubation with the mutant gp120 ΔHXα-1, and

d) bonding of the Leu3a l antibody to the CEMs.

On examination of these results, it may be observed that the gp120 wt inhibits more than 98% of Leu3a bonding (graph b of FIG. SA). In contrast to this, gp12O ΔHXα-1 does not modify the bonding of the antibody (<5.6%, graph c of FIG. SA).

ELISA cell method

The effect of the gp12O on the bonding of the monoclonal antibody F101.69 is studied by the ELISA cell method by proceeding as follows :

U-shaped micro-titration plates (Nunc), Maxisorb® are saturated with PBS/3% BSA, for 30 minutes, at 37° C., and incubated with 25 μl of a 1:1 mixture (10 μg/ml) of a monoclonal anti-CD5 antibody (F145,6F3) and anti-CD100 (F93, 7G2) for 16 hours at 4° C.

After several washings, 10⁵ CEM cells are distributed in each well, before centrifugation of the plate ac 900 g for 5 minutes, then incubation for 30 minutes at 37° C., and 2 washings with 200μl of PBS/0.3% BSA per well.

The wells, using four specimens, are incubated with the soluble gp120 proteins for 1 hour, at 37° C., and washed twice in PBS/0.3% BSA.

For the CD4 inhibition experiments, the cells are then Incubated for 30 minutes at 37° C., with a dilution of 1/1000 of monoclonal antibody F101.69 anti-CD4 coupled with peroxydase.

As a variant, after incubation with the gp120 proteins, SDF-1α is added at a concentration of 10 μg/ml, for 30 minutes, at 20° C. The wells are washed twice with PBS/0.3% BSA and then incubated with a biotinylated goat antibody, anti-SDF-1α in PBS/3% BSA/0.02% sodium azide and then developed with a streptavidine-biotin-peroxydase complex at 20° C., for 30 minutes.

The optical density (OD) is measured at 492 nm on a Labsysteme Multiscan RC spectrophozometer. The cell-ELISA plate includes two internal standards, without gp120 and without SDF1α.

The experimental values are expressed as a percentage inhibition of the corresponding reference values.

The reference ODs vary from 1.0 to 1.5.

The wells without cells, saturated with PBS/3% BSA, indicate that the non-specific bonding of the recombinant gp12O proteins is less than 5%.

Similar results are obtained with the conjugated anti-CD4 F101.69-PE antibody and confirmed by the cell-ELISA method (FIG. 5B) with similar results whether sodium azide is present or not. Study of the effect of the gp120 on the bonding of other monoclonal anti-CD4 antibodies by FACS

Equivalent studies carried out with different monoclonal anti-CD4 antibodies directed against distinct sites of the CDR2 loop of D1 in CD4, show that the gp12O ΔHXα-1 does not interfere with the ability of these antibodies to bond to the receptor CD4. In effect, the bonding of anti-CD4 monoclonal antibodies, Leu3a , F101.69, 1388.2, ST40, BL4, OKT4 and BF5, is also controlled after incubation of the CEM cells with the gp wt or the soluble gp12O ΔHXα-1 (10 μg/ml) for 1 hour at 37° C.

The cells are washed (2 washings in PBS/0.3% BSA) and colored with the anti-mouse, IgG-specific conjugated FITC (100 μg/ml) before carrying out the flow cytometry analysis, as described in Example 2 above, in the presence of or in the absence of sodium azide with 0.2% in the washing and antibody solutions.

In these tests, the soluble gp12O (gp120 wt or gp120 ΔHXα-1) is used, in a volume of 50 μl at different concentrations.

These results are given in Table 1. All the anti-CD4 antibodies have been detected with an anti-mouse goat antiserum marked with FITC, except for the 13B8.2 which was conjugated with PE.

TABLE 1

| | Effect of the gp120 on the bonding of | | |
|---|---|---|---|
| | the AcM anti-CD4 | | |
| | Mean fluorescence Intensity | | |
| | Pre-incubation | | |
| Antibodies | Medium only | gp120 wt | gp120 ΔHXα-1 |
| 13B8.2 | 1054.78 | 73.13 | 940.74 |
| ST40 | 416.00 | 87.73 | 360.16 |
| BL4 | 489.38 | 93.40 | 418.96 |
| OKT4 | 526.53 | 481.65 | 530.00 |
| BF5 | 430.00 | 410.00 | 424.31 |

It emerges from these results that the gp12O ΔHXα-1 is incapable of bonding itself to the receptor CD4.

Considering these results, the ability of the gp12O to bond itself to the surface of CEM cells in relation to their concentration was verified.

The FACS analysis has been carried out with the cells in the presence of sodium azide (0.2%) to prevent possible internalization of the target receptors.

Under these conditions, as shown in FIG. 5C, the gp12O ΔHXα-1 bonds itself to the surface of the CEM cells in a dose dependent way, but at lower levels than the gp12O wt.

When these latter experiments were carried out in the absence of sodium azide, no bonding of the gp12O ΔHXα-1 onto the CEM cells was observed.

It will be noted with interest that the absence of sodium azide does not modify the ability of the gp12O wt to inhibit the bonding of the monoclonal anti-CD4 antibodies.

FIG. 5D illustrates the experiment corresponding to FIG. 5C but enables one to verify the saturation doses.

Study of the inhibition of the bonding of SDF1-α to CXCR4

The bonding of the gp12O ΔHXα-1 to the CEM cells lead one to determine if the association with the chemokine receptor CXCR4 could be responsible for the phenomenon reported above.

To this end, it was investigated if the gp120 wt or the gp120 ΔHXα-1 could inhibit the bonding of SDF1-α.

FIGS. 6A and 6B give the results respectively of the FACS and cell-ELISA analyses carried out.

FIG. 6A relates to a test with coloration using a biotinylated monoclonal anti-SDF1-α antibody developed with a streptavidine-PE conjugate. The CEM cells are incubated with SDF1-α alone (10 μg/ml) or are incubated beforehand with gp120 wt or ΔHXα-1, at concentrations, respectively of 10 μg/ml and 30 μg/ml.

As a control sample, CEM cells are used which have not been exposed to SDF1-α.

The tests corresponding to the results in FIG. 6B are carried out in the presence of various concentrations of gp12O wt and of ΔHXα-1 mutant by applying the cell-ELISA method.

It is observed that the bonding of SDF1-α (10 μg/ml) reduces to 44.4% and 36% of the control value in the presence respectively of 10 μg/ml and 30 μg/ml of gp120 wt (FIG. 6A).

After pre-incubation with the gp12O ΔHXα-1 at a concentration of 10μg/ml and 30 μg/ml significant falls are also observed respectively to 35.3% and 25% in relation to the control.

Similar results are obtained using the cell-ELISA method as represented by the respective inhibition levels (FIG. 6B).

A specific interaction between .CXCR4 and gp120 wt and gp12O ΔHXα-1 is therefore observed.

Study of the inhibition of the bonding of antibody anti-CXCR4

The ability of the gp120 wt and the gp12O ΔHXα-1 to inhibit the bonding of the monoclonal 12G5 antibody, anti-CXCR4 was also evaluated.

The antibody is used at a rate of 10 μg/ml. The measurements are carried out after pre-incubation with the gp12O wt or the mutant ΔHXα-l or SDF1-α. The results are given in Table 2 and expressed as a percentage of the total bonding taken as 100%.

TABLE 2
Effect of the gp120 on the capacity for bonding of a
AcM anti-CXCR4

| | Capacity for bonding (%) of 12G5 to the | | | | |
|---|---|---|---|---|---|
| | ligand cells at different concentrations | | | | |
| | (μg/ml) | | | | |
| | | | CHO-K1 | | |
| Pre-incubation | CEM CD4^{-a} | | CXCR4⁺/CD4^{-o} | | |
| | 10 | 40 | 90 | 2 | 10 |
| SDF-1α | 20.7 | ND | ND | ND | ND |
| gp120 wt | 59.5 | 58 | ND | 75.0 | 64 |
| gp120 ΔHXα-1 | 80.0 | 77.2 | 44.8 | 74 | 44.8 |

a) The capacity for bonding of the AcM 12G5 anti-CXCR4 (10 μg/ml) to the CEM cells is determined after pre-incubation with either gp120 wt, gp12O ΔHXα-1, or SDF-1α and is presented as the percentage of total bonding (100%) ;

b) The capacity for bonding of the AcM 12G5 (10 g/ml) to the CHO—Ki CXCR4- CD4- cells is determined after pre-incubation with the soluble gp12O wt or the gp12O 15 ΔHXα-1, and is presented as the % of total bonding (100%)

c):ND=Not Determined.

It is observed that the gp120 inhibit the bonding of the antibody with about 80% and 60% bonding in 20 relation to concentrations, respectively of 10 and 40 μg/ml and less than 45% at a concentration of 90 μg/ml of gp120 ΔHXα-1.

The bonding of the monoclonal antibody 12G5 after incubation with SDF-1α causes a fall to 20% bonding with respect to the CEM cells to that of the control.

The similar experiments carried out on the CHO—KI CXCR4⁻/CD4⁻show that gp120 wt and gp12O ΔHXα-1 inhibit the bonding of the AcM 12 GS respectively of 25 and 36% at concentrations of 2 and 10μg/ml.

Study of the bonding of the surface of CD4 negative cells

Next, it was verified whether the gp12O ΔHXα-1 is capable of bonding to the surface of CD4-negative cells expressing in a new manner, the receptor CXCR4 (CHO—K1 cell lineage).

The glycoproteins of gp120 (2 μg/ml) are incubated for 4 hours, at 4° C., with CD4 negative cells (CHO-K1) The cells are incubated with human antibodies anti-gp120 and are then washed and colored with an anti-human conjugate IgG-PE or -FITC and the cells are treated by flow cytometry analysis as described in Example 2 above.

The inhibition of the bonding of SDF1-α is determined after incubation of the CEM cells with various concentrations of gp12O wt and gp120 ΔHXα-1 for 1 hour at 37° C. in PBS/3% BSA.

SDF1-α(10 μg/ml) is then added into PBS/3% BSA during 30 minutes at 37° C. The cells are colored with a biotinylated polyclonal goat antibody, an anti-human anti-SDF1-α, in PBS/3% BSA/0.2% sodium azide for 30 minutes at ambient temperature.

The streptavidine-PE conjugate is added during 30 minutes at ambient temperature in PBS/3% BSA/0.2% sodium azide before analysis by flow cytometry.

The inhibition of the bonding of the monoclonal antibody 12G5 anti-CXCR4 is determined after incubation of the CAM cells with various concentrations of gp120 wt, gp12O ΔHXα-1, or 10 μg/ml of SDF-1α for 30 minutes at 37° C.

The accessibility of the CXCR4 is controlled by addition of monoclonal antibody 12G5 (10 μg/ml) in the presence of 0.2% sodium azide during 1 hour at 4° C.

The cells are washed twice with PBS/0.3% BSA/0.2% sodium azide, and colored with an anti-mouse IgG antibody, conjugated to FITC in PBS/3% BSA/0.2% sodium azide, before the analysis by flow cytometry.

The results are given in FIG. 7A where curve a) corresponds to the fluorescence base, curve b) relates to the bonding of gp120 wt (2 μg/ml) to the cells and curve c) to the bonding of the gp12O mutant.

The bonding of the gp12O wt and the gp120 ΔHXα-1 to the CHO—K1 cells has been determined using an anti-HIV-1 IgG and developed with an anti-human IgG marked with PE.

Bonding is clearly observed with a mean intensity of fluorescence that reaches 11.92 and 13.92 respectively for the gp120 wt and the gp12O ΔHXα-1 while the intensity of the control, obtained in the absence of gp120 is 3.35.

Similar, low, mean fluorescence intensities are obtained after incubation of the CHO—K1 cells, CXCR4⁻/CD4⁻ with concentrations of 2, 10 and 30 μg of gp12O wt or mutant per ml (FIG. 7B).

Example 4: Study of the inhibition of the infectivity of HIV-1 by the soluble recombinant gp120 ΔHXα-1

In order to study the ability of the gp120 ΔHXα-1 to inhibit infection by HIV-1, pre-Incubation of the HeLa 24 cells is carried out with the soluble protein, at 4° C., for 1 hour, before exposing them to the HIV-1 virus LAI.

The tests are carried out as follows An infection is brought about 24 hours after seeding of 10⁴ HeLa-P4 cells (CD4+, LTR-LacZ)/well, in micro-titration plates with 96 wells.

The cells are then pre-incubated, under moderate agitation, in a DMEM medium, lacking in serum, in the presence of different concentrations of gp12O wt or soluble recombinant gp12O ΔHXα-1, for 1 hour, at 4° C. They are then incubated with 25 μl of ultra-centrifuged infectious particles of HIV-1 LAI produced from infected CEM cells.

The induction of β-galactosidase activity, that is to say product of the LacZ gene from the HeLa P4 reflects the transactivation Tat and, as a consequence, the infection by HIV-1.

After 24 hours, the β-galactosidase activity is measured in the cellular lysats of the wells used in four examples.

To this end, the cells are lysed in 100 μl of a buffer containing 0.125% of NP40, 60 mM of Na₂HPO₄, 40 mM of NaH₂PO₄, 50 mM of β-mercapto-ethanol, 25 mM of EDTA, 10 mM of KC1, 10 mM of MgSO₄ and 100 μl of 80 mM of sodium phosphate at pH 7.4, 10 mM of MgCl₂, 10 mM β-mercapto-ethanol before the addition of 6 mM of chlorophenol red-monosodium salts of β-galactopyranoside.

The mixture is incubated for 30 minutes at 37° C. and the absorbency is measured at 574 nm.

The results obtained show viral inhibition of 50% (IC₅₀) after pre-incubation with one or the other of the gp12O wt or gp12O ΔHXα-1 at a concentration of 100 nM (FIG. 8).

Hence, despite its absence of interaction of the gp120 ΔHXα-1 with CD4, this molecule is capable of inhibiting a viral infection by HIV-1, apparently by interaction with the secondary receptor CXCR4.

1. Glycoprotein mutants of HIV-1 gp120 envelopes, characterized in that they are glycoproteins capable of interacting specifically with the chemokine receptors and have an inhibiting activity with respect to an HIV-1 infection independent from CD4.

2.Mutants according to claim 1, characterized in that the deleted region corresponds to an α helix structure, such as is present in the native glycoproteins of envelope.

3. Mutants according to claim 1 or 2, characterized in that the deleted region corresponds to the α-1 helix structure such as is present in the Cl region of the native gp120 of HIV-1.

4. Mutants according to claim 3, characterized in that the deleted region corresponds to the fragment of sequence E61 to S85, such as is present in the Cl region of the native gp120 of HIV-1.

5. Mutants according to any one of claims 1 to 4, characterized in that the glycoproteins are recombinant.

6. Antibodies, characterized in that they are directed against the mutants according to any one of claims 1 to 5.

7. Application of the mutants according to any one of claims 1 to 5 as prototype viral envelopes for the study of their immunogenic power.

8. Application of the mutants according to any one of claims 1 to 5 as competitors of anti-viral drugs or antibodies at the virus receptor.
